(19) United States
(12) Patent Application Publication
(10) Pub. No.: US 2012/0220003 A1
(43) Pub. Date: Aug. 30, 2012

(54) METHOD FOR CONTINUOUSLY SEPARATING ORGANIC MATERIALS OF INTEREST FROM FERMENTATION

(75) Inventors: Joel Schwartz, Caluire (FR); Philippe Marion, Vernaison (FR)

(73) Assignee: Rhodia Operations, Aubervilliers (FR)

(21) Appl. No.: 13/394,329

(22) PCT Filed: Sep. 3, 2010
(86) PCT No.: PCT/EP10/62933

§ 371 Date: May 18, 2012

(30) Foreign Application Priority Data
Sep. 3, 2009 (FR) 0956006

(51) Int. Cl. C12P 7/16; C12M 1/12

(52) U.S. Cl. 435/160; 435/289.1

(57) ABSTRACT

A method for continuously separating organic materials of interest from fermentation, in particular lactic or alcoholic fermentation, by flash evaporation is described. The method can make it possible to avoid inhibiting the fermentation reaction and to obtain high yields and productivity.

The present invention relates to a process for continuously separating organic products of interest from fermentation, in particular lactic or alcoholic fermentation, by flash evaporation. Said process in particular makes it possible to avoid inhibiting the fermentation reaction and to obtain very high yields and productivity.

### PRIOR ART

Industrial fermentation, in particular lactic or alcoholic fermentation, processes are well known and make it possible to produce organic products of interest which can be used for various applications.

The problem of the isolation of the organic products of interest resulting from the fermentation, and in particular their separation from the biomass, often arises.

It is in particular possible to carry out liquid/liquid extractions or separations using an absorbent such as active carbon. However, for liquid/liquid extraction, it is necessary to find a solvent which has a very good coefficient of partition between the aqueous phase and the organic phase, and if it is desired to recycle the aqueous phase to the fermenter in order to improve yield and productivity, it is necessary for this solvent not to be an inhibitor of the fermentation and, if possible, to be of very low solubility in the aqueous phase. This remains rarely the case. For treatment with an adsorbent, it is once again necessary for the thermodynamics to be very favorable and for the adsorbent not to give rise to toxic substances. It is, moreover, necessary to consider the desorption and washing phases; this solution is often complex for industrial-scale use.

It would also be possible to carry out stripping of the organic products of interest, in particular with the noncondensable products generated during fermentation, such as carbon dioxide, methane and hydrogen. However, this proves to be very complex since the fermentation temperature is in the region of 30 or 40° C., and, at this temperature, it is strictly impossible to entirely and simply strip the organic compounds and the water with the noncondensable products generated during fermentation. It would therefore be necessary to recycle these noncondensable products using a large gas compressor, stirring systems in complex fermenters for dispersing these gases, and disproportionate condensation systems with refrigeration units so as to efficiently trap the organic compounds. Furthermore, this is without increasing the temperature or decreasing the pressure in the fermenter so as not to damage the microorganisms and reduce the yield of the reactions of interest.

There was thus a need to develop a reliable and industrially realistic process for efficiently separating the organic products of interest resulting from fermentation while at the same time avoiding the drawbacks mentioned above.

### Invention

The applicant has developed a process for separating the organic products of interest resulting from fermentation, consisting in continuously withdrawing fermentation must from the fermenter so as to subsequently separate (strip) the organic compounds of interest in an apparatus dissociated from the fermenter, then in particular recycling the heels from the separator to the fermenter. This is in particular carried out by reducing the pressure of the must in a flash chamber working under vacuum. The organic compounds are flashed and carried over with water; as flash-chamber heels, the must cooled by evaporating off the solvents and the water is recycled to the fermenter at a temperature such that it absorbs the fermentation exotherm. In the flash chamber, the pressure is regulated so as to obtain the temperature which gives the fermenter thermal equilibrium. Heat is supplied to the flash as required.

In order to avoid the drawback of the fragility of the microorganisms which may rupture at the time of the flash, the biomass is separated before the flash, for example by ultrafiltration, the retentate being sent back directly to the fermenter, the filtrate to the flash.

This technical solution thus makes it possible to avoid introducing noncondensable products into the fermenter and therefore to avoid using a compressor or a complex stirring device in the fermenter. This also makes it possible to avoid the use of a heat exchanger in the fermenter, since the thermal equilibrium is provided by the recycling of the cooled flash heel, and therefore to simplify the technology of the fermenter, which no longer requires an internal exchanger or a stirring system. This method in particular makes it possible to avoid inhibiting the fermentation reaction and to obtain very good yields and productivity.

The main subject of the present invention is thus a process for continuously separating organic products from fermentation in a fermenter, comprising at least the following steps:
a) removal of a part of the must from the fermenter during fermentation;
b) separation of the biomass, which is sent to the fermenter;
c) flash evaporation of the liquid separated in step b), and obtaining of the organic products in the gas phase; and
d) isolation of the organic products.

The process is preferentially carried out continuously or batchwise.

The term “fermentation” is intended to mean a biochemical reaction which converts the chemical energy contained in a carbon source, in particular glucose, into another form of energy that can be directly used by the cell, in particular in the absence of dioxygen. The fermentation according to the invention concerns in particular the lactic fermentation and preferentially the alcoholic fermentation which is the result of a metabolic chain which converts fermentable sugars, in particular by means of yeasts, into alcohol and carbon dioxide with the release of heat. The fermentation according to the invention results in the production of the organic products “of interest”.

The term fermentation “must” is intended to mean the reaction medium comprising in particular the biomass, the fermentable products and the organic products obtained by means of the fermentation.

The term “biomass” is intended to mean all of the living matter, generally the microorganisms, present in the fermentation medium.

The fermenter used according to the present invention is a technological unit in which microorganisms, such as yeasts, bacteria, microscopic fungi, algae, or animal or plant cells, are multiplied for the bioconversion of an organic molecule of interest. This bioreactor generally makes it possible to control the culture conditions such as the temperature, the pH and the aeration. In the fermenter, the temperature is generally between 30 and 45° C.

The separation of the biomass is generally carried out in step b) before carrying out the flash evaporation of step c). Filtration, ultrafiltration, decanting, centrifugation and/or ultracentrifugation can, for example, be carried out. It is also possible to carry out the separation of the biomass in the gas-liquid separator used to carry out the flash evaporation, in particular by placing a barometric height of liquid in the gas-liquid separator so as to avoid the shock of depressurization of the microorganisms during the flash evaporation. In this case, the entry of the must into the system for the flash evaporation preferentially takes place in a zone that has a pressure equivalent or equal to the pressure of the fermenter. The barometric height may allow a pressure gradient of between 0.5 and 1 bar.

The circulation of the must can, for example, be carried out by means of a pump.

The liquid obtained, also called liquid separated in step b), corresponds to the must as defined above, essentially freed of the biomass. This liquid generally has the same temperature as that of the fermenter.

In the case of the ultrafiltration particularly preferred according to the invention, the filtrate liquid is conveyed to the flash evaporation step and the residue or retentate is sent back to the fermenter. Use is generally made of systems of tangential ultrafiltration on an external circulation loop under pressure. These ultrafiltration systems, which generally have a cut-off threshold of about 0.01 μm, normally operate under transmembrane pressures TMPs of about from 1 to 3 bar. The proportions of such an ultrafiltration system depend on the amount of organic products to be extracted from the reactor and on the time for which this extraction must be carried out. These parameters determine the volume of filtrate to be produced with a view to a flash evaporation. The volume of filtrate to be produced and the time for which the filtrate must be produced, i.e. the operating time of the ultrafiltration system, determine the surface area of membrane to be installed. The filtration flow rates are usually between 20 and 100 l/m².h. according to the characteristics of the fermentation must and the TMP applied. The circulation speed of the must inside the ultrafiltration module is generally between 2 and 5 m/s. The membranes used are generally of tubular inorganic type having the advantage of being able to undergo effective cleaning treatments and of being not very sensitive to clogging.

The flash evaporation of step c), or flash vacuum-expansion, consists in placing the liquid in a vessel under vacuum, in particular with a pressure of between 0.001 and 0.9 bar. The placing under vacuum makes it possible to decrease the saturation temperature and to evaporate off a part of the liquid. The evaporation will take place by taking energy from the mixture and thus cooling it. The process of flash evaporation under vacuum has been very widely used for several decades in various industries. It is found in various applications, such as the desalination of seawater, the concentration and pasteurization of milk or the treatment of waste water loaded with soluble oils. To this effect, it is possible to use, for example, a gas-liquid separator such as an expansion vessel or expansion chamber, also called a flash chamber. It is also possible to use one or more expansion vessels in particular at different pressures. A heat exchanger can be added in or next to the gas-liquid separator, according to the needs in terms of energy provision, in order to heat or cool the flash evaporation medium. A tubular or plate heat exchanger can, for example, be used.

The gas-liquid separator is generally a cylinder with a vertical axis. It comprises, for example, a feed pipe for the liquid from the fermenter, an outlet at the bottom and an outlet at the top. The column height may depend on the barometric height desired, as explained previously.

The gas-liquid separator may comprise packing or plates so as to have some theoretical separation stages.

According to the present invention, the flash evaporation is preferentially carried out at a pressure of between 10 and 200 mbar.

The temperature during the flash evaporation is generally between 10 and 40° C.

The vaporization rate may be between 1 and 70% by weight, preferentially between 30 and 60% by weight.

This separation is preferentially carried out without the addition of a particular solvent outside the compounds resulting from the fermentation.

The gas phase resulting from the flash evaporation thus comprises the organic products of interest and also dissolved noncondensable products such as CO₂, H₂ and CH₄.

The gas phase is preferentially brought to a condensation means, such as a condenser, in order in particular to separate the residual noncondensable products from the organic products of interest.

The aqueous phase resulting from the flash evaporation generally comprises large amounts of water and a minority of organic compounds. This aqueous phase is preferentially recycled to the fermenter, and is in particular at a temperature that is below the fermenter temperature, generally a temperature that is from 5 to 20° C. below the fermenter temperature.

The organic products of interest of the gas phase are then isolated by means of one or more separation techniques known from the prior art, such as, in particular, by distillation, liquid-liquid extraction, crystallization and/or adsorption. When the boiling temperatures are very close, it may be advisable to use a process of fractional distillation which consists of several successive refinement steps. It is also possible to introduce a part of the distillate at the top of the column in the case of continuous distillation, in order to improve the purity of the vapor phase.

Some vapors of the gas phase can in particular be recondensed and the condensates can optionally be reintroduced into the fermenter. A part or all of the water of the gas phase resulting from the flash separation can be condensed and recycled to the fermenter. The water necessary for carrying over all the organic products produced in the fermenter is generally recycled. To this effect, tube, plate or spiral condensers can in particular be used.

The process according to the present invention is particularly suitable for separating the biobutanol resulting from the fermentation of sugar cane molasses. In this case, the gas phase of step c) generally comprises CO₂, H₂, butanol/water heteroazeotrope, water, acetone and ethanol, the liquid phase of the flash essentially consisting of water. The process according to the present invention is also suitable for separating the ethanol resulting from fermentation.

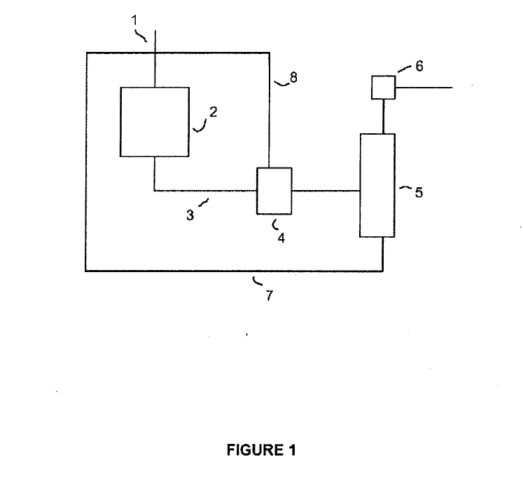

The present invention also covers devices for implementing the process according to the invention. A preferred device corresponds to that of FIG. 1 and comprises in particular a fermenter (2) which has a feed for nutrients, gas, musts and the like (1) and a means for discharging the must (3). The must is conveyed to a means for separating the biomass (4), such as an ultrafiltration module. The biomass is sent back to the fermenter (2) via a suitable conveying means (8). The fermentation liquid freed of its biomass is sent to the flash evaporation system (5), such as a gas-liquid separator (5). The liquid phase is conveyed to the fermenter (2) via a suitable means (7) and the gas phase is then discharged to a condenser (6) which separates the residual noncondensable products from the organic products of interest. The latter will then be isolated by means of one or more conventional separation techniques. At the outlet of the condenser (6), a part of the liquid can be refluxed to the top of the gas-liquid separator (5).

A specific language is used in the description so as to facilitate understanding of the principle of the invention. It should nevertheless be understood that no limitation of the scope of the invention is envisioned through the use of this specific language. Modifications, improvements and optimization can in particular be envisioned by a person familiar with the technical field concerned, on the basis of their own general knowledge.

The term “and/or” includes the meanings “and” and “or” and also all the other possible combinations of components connected with this term.

Other details or advantages of the invention will appear more clearly in the light of the examples given below solely by way of indication.

### Experimental Section

The device tested corresponds to that of FIG. 1 and comprises in particular a fermenter (2) which has a volume of 1000 m³ and operates at a temperature of 38° C. The fermenter comprises concentrated green juice from sugar cane at the beginning of fermentation. The separating means (4) used is a tangential ultrafiltration module. The flash evaporation system is a gas-liquid separator (5) of flash chamber type. The liquid phase is conveyed to the fermenter (2) via a suitable means (7) and the gas phase is then discharged to a condenser (6). A part of the condensed water from the gas phase, freed of the organic products, is conveyed to the fermenter for recycling. The production cycle is 200 hours. The ultrafiltration outlet flow rate is regulated in such a way that, for example, 90% of the mass is recycled to the fermenter via the means (8) and 10% of the mass passes through the membranes of the ultrafiltration module (4). The flash separation is carried out at a temperature of 30° C. and a pressure in the gas phase of 0.045 bar.

With a vaporization rate of 40 mol % or 50% by mass in the flash chamber, the following streams entering and leaving the flash evaporation are obtained:

| | Liquid | | Liquid |
|---|---|---|---|
| Products | entering | Gas leaving | leaving |
| Butanol | 5 | 8.6 | 0.9 |
| Acetone | 1.6 | 3.6 | less than |
| | | | 0.001 |
| Ethanol | 0.1 | 0.2 | less than |
| | | | 0.001 |
| Water | qs | qs | qs |

% by weight

A production of 1000 kg/h of butanol in the fermenter and of 1000 kg/h in the stream leaving the condenser (6) is obtained.

Good separation of the organic products of interest in the gas phase is thus observed, with a good yield, while at the same time ensuring a thermal equilibrium with the recycling of the flash heel to the fermenter making it possible to avoid inhibiting the reaction in the fermenter.

1. A process for continuously separating organic products from fermentation in a fermenter, the process comprising at least the following steps:
a) removing a portion of must present in the fermenter during fermentation, wherein the must comprises a biomass and a liquid comprising water and organic compounds;
b) separating the biomass from the liquid in the portion of the must removed from the fermentor and sending the separated biomass to the fermenter;
c) flash evaporating the liquid separated, from the biomass and separating organic products in the gas phase from the water and forming an aqueous phase; and
d) isolating the organic products.

2. The process as claimed in claim 1, wherein separating the biomass from the liquid in the portion of the must removed from the fermentor is carried out before flash evaporating the liquid separated from the biomass.

3. The process as claimed in claim 1, wherein the separation of the biomass in step b) is carried out by filtration, ultrafiltration, decanting, centrifugation and/or ultracentrifugation.

4. The process as claimed in claim 1, wherein separating the biomass from the liquid in the part of the must removed from the fermentor is carried out in a gas-liquid separator that is used to carry out the flash evaporation.

5. The process as claimed in claim 1, wherein flash evaporating the liquid separated from the biomass is carried out at a pressure of between 10 mbar and 200 mbar.

6. The process as claimed in claim 1, wherein the temperature during flash evaporating the liquid separated from the biomass is between 10° C. and 40° C.

7. The process as claimed in claim 1, wherein during flash evaporating the liquid separated from the biomass, the vaporization rate is between 1% by weight and 70% by weight.

8. The process as claimed in claim 1, wherein flash evaporating the liquid separated from the biomass is carried out without the addition of a solvent where the solvent is different from compounds produced from the fermentation.

9. The process as claimed in claim 1, wherein the aqueous phase resulting from the flash separation is at a temperature that is below the fermenter temperature and is recycled to the fermenter.

10. The process as claimed in claim 1, wherein the water aqueous phase resulting from flash separation is recycled to the fermenter.

11. The process as claimed in claim 1, wherein the must comprises sugar cane molasses and the organic product isolated comprises butanol.

12. A device for implementing the process as claimed in claims 1.

13. The device as claimed in claim 12, comprising a fermenter having a feed for nutrients, gas and must and a means for discharging the must; a flash evaporation system comprising a condenser; a means for separated the biomass from fermentation liquid in the must; a means for conveying the must to a means for separating the biomass from the liquid in the must, a means for conveying the biomass separated from the liquid in the must to the fermenter, a means for conveying the fermentation liquid separated from the biomass to the flash evaporation system, a means for conveying the fermentation liquid the fermenter and a means for discharging a gas phase formed in the flash evaporation system to the condenser.
